(19) United States
(12) Patent Application Publication
(10) Pub. No.: US 2021/0353798 A1
(43) Pub. Date: Nov. 18, 2021

(54) UNIVERSAL MASK CARRIER AND DECONTAMINATION DEVICE

(71) Applicants: Charlotte Rosalind Spear, Morgantown, WV (US); Jeffry Skiba, Chandler, AZ (US); Campion Acheson Platt, West Palm Beach, FL (US)

(72) Inventors: Charlotte Rosalind Spear, Morgantown, WV (US); Jeffry Skiba, Chandler, AZ (US); Campion Acheson Platt, West Palm Beach, FL (US)

(21) Appl. No.: 17/319,737

(22) Filed: May 13, 2021

Related U.S. Application Data
(60) Provisional application No. 63/024,138, filed on May 13, 2020.

(51) Int. Cl. A61L 2/10; A45F 5/02

(52) U.S. Cl. CPC A61L 2/10; A61L 2/26; A45F 5/021

(57) ABSTRACT

A mask carrying system comprising a housing that can have a front portion detachably coupled to a back portion, and an outer surface and an inner surface defining an exterior and an interior of the housing. A mask carrying system can further comprise a disinfectant to disinfect an item on the interior of the housing to a 2-log reduction of at least one pathogen. The disinfectant can be a plurality of ultraviolet lights illuminating the interior of the housing. The front portion can substantially conform to a mask shape. The connector plate can be coupled to a power source wherein the power source supplies power to the disinfectant.

### CROSS-REFERENCE TO RELATED APPLICATIONS

This application claims benefit from currently pending U.S. Provisional Application No. 63/024,138 titled “Universal Mask Carrier and Decontamination Device” and having a filing date of May 13, 2020, all of which is incorporated by reference herein.

### FIELD OF THE INVENTION

The present specification relates to a masking carrying device, more particularly a universal mask carrying device that can carry and sanitize a mask while being carried.

### BACKGROUND OF THE INVENTION

With the COVID-19 outbreak, healthcare workers have become acutely aware of the increased need to use personal protection equipment (“PPE”) to protect frontline workers, patients, and the general public. N95 masks and other type of masks in general are the mainstay of protection against viral particle transmission. As a result, masks which are traditionally single-use, disposable products are being reused time and time again, which serves as a great risk to health workers and patients. Little is known about viral transmission of COVID-19 through the masks, however, studies do support that healthcare workers often apply the masks incorrectly and in such a way which causes contamination to the inner part of the mask, as well as contamination of the hands. Both of these put not only the mask wearer at risk, but also anyone with whom they are in contact.

Despite proper training on mask application, healthcare workers are carrying COVID-19-laden masks in the workplace in their pockets and on their person where the external surface is in contact with patients and other workers. This mask must be applied without coming into contact with the mask exterior, which is extremely challenging, if not impossible. This life-threatening problem poses an acute issue for infection control. Although we are still learning about the viral transmission, the viral load seems to be the dominant factor with the mask exterior having the highest density of viral particles. One possible way to prevent exposure would be to isolate the exterior surface of contaminated N95 masks thereby preventing inadvertent transmission.

Therefore, there is a need for a device that can carry any type of mask when not in use and decontaminate the mask when carried within the carrying or storage device.

### BRIEF SUMMARY OF THE INVENTION

A mask carrying system comprising a housing that can have a front portion detachably coupled to a back portion, and an outer surface and an inner surface defining an exterior and an interior of the housing. A mask carrying system can have at least one loop coupled to the back portion that allows the housing to be removably attached to a belt. A mask carrying system can further comprise a disinfectant to disinfect an item on the interior of the housing to a 2-log reduction of at least one pathogen. A connector plate can be coupled to the outer surface of the front portion of the housing. The disinfectant can be a plurality of ultraviolet lights illuminating the interior of the housing. The disinfectant can be a heat source that heats the interior of the housing to at least 167 degrees Fahrenheit for at least 30 minutes.

The front portion can substantially conform to a mask shape. The connector plate can be coupled to a power source wherein the power source supplies power to the ultraviolet lights. The loop can be a belt clip or a belt loop. The front portion can be coupled to the back portion by a zipper, at least one magnet, or a hinge and clasp. The power source can comprise an on/off switch and a charging port for the user to recharge the power source. The belt can be the user's own belt or a customized belt that is attached to the loop. The front portion can further comprise a mask strap protrusion wherein the mask strap protrusion holds the straps of a mask.

A method of disinfecting an item, the method comprising the acts of providing a housing having a front portion detachably coupled to a back portion, and an outer surface and an inner surface defining an exterior and an interior of the housing. Placing the item into the interior of the housing. Coupling the front portion to the back portion. Disinfecting the interior of the housing sufficient to provide to a 2-log reduction of at least one pathogen on the item. The act of disinfecting comprises illuminating the item with ultraviolet light. The act of disinfecting comprises heating the interior of the housing to at least 167 degrees Fahrenheit for at least 30 minutes.

The front portion substantially conforms to a mask and further comprises placing the mask within the housing to align the shape of the mask to the contour of the front portion. Carrying the housing coupled to a belt on a user. The act of disinfecting the interior of the housing comprises supplying at least one of ultraviolet light, heat, and a chemical disinfectant. Calibrating the amount of ultraviolet light, heat, and chemical disinfectant to provide a log-2 reduction of at least one pathogen. Docking the housing onto a docking station and recharging a power source which supplies the ultraviolet light or heat with power. Sending a notification to a user via a portable computing device when the mask is decontaminated.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for” and will also recite the word “function” (i.e., will state “means for” performing the function of molding a fishing lure, without also reciting in such phrases any structure, material or act in support of the function. Thus, even when the claims recite a “means for performing the function of molding a fishing lure, step for performing the function of molding a fishing lure,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 is an isometric view of a mask carrying system in accordance to one, or more embodiments;

FIG. 2 is an isometric of a mask carrying system of another embodiment in accordance to one, or more embodiments;

FIG. 3 is a side view of a mask carrying system of the same embodiment of FIG. 2 in accordance to one, or more embodiments;

FIG. 4 is a isometric view of a mask carrying system of another embodiment in accordance to one, or more embodiments;

FIG. 5 is a side view of a mask carrying system of the same embodiment of FIG. 4 in accordance to one, or more embodiments;

FIG. 6 is a bottom view of a mask carrying system of the same embodiment of FIG. 4 in accordance to one, or more embodiments;

FIG. 7 is a back view of a mask carrying system of the same embodiment of FIG. 4 in accordance to one, or more embodiments; and

FIG. 8 is a back isometric exploded view of a mask carrying system of the same embodiment of FIG. 4 in accordance to one, or more embodiments; and

FIG. 9 is a back view of a mask carrying system in accordance to one, or more embodiments.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

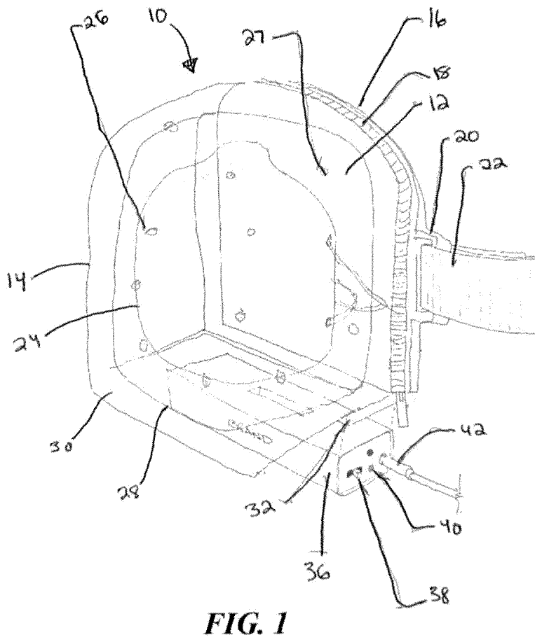

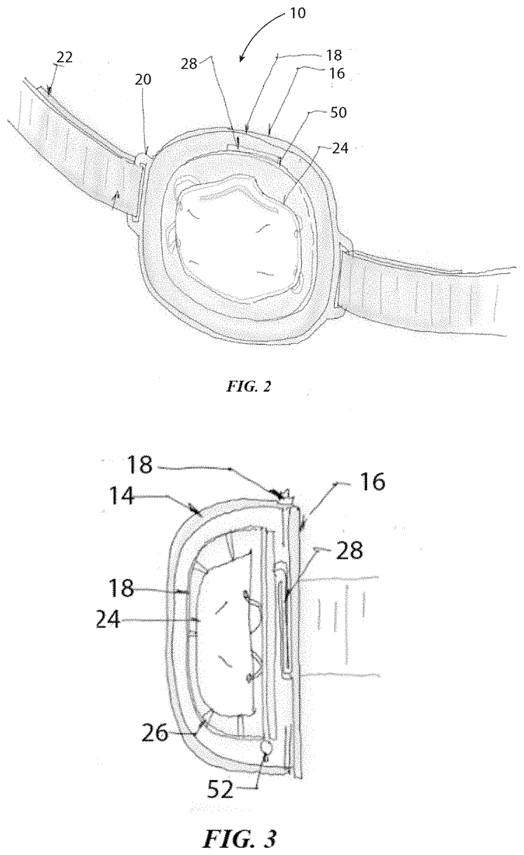

Referring to FIG. 1 a mask carrying system 10 for carrying and decontaminating a mask 24 comprising a housing 12 having a front portion 14 and a back portion 16 wherein the back portion can be attached to the front portion by an attachment mechanism 18 or the front portion can be detachably coupled to a back portion, and an outer surface 30 and inner surface 28 defining an exterior and interior of the housing. The outer surface 30 can have a connector plate 32 coupled to the outer surface. The connector plate 32 can be attached by any suitable means but in the preferred embodiment the connector plate can be attached by for example, rivets, buttons, hook and loop fastener, glue, or the like. The connector plate 32 can be manufactured from, for example, stainless steel, aluminum, plastics, or any other suitable rigid or non-rigid material. The connector plate 32 can be either permanently attached or removably attached to the front portion 14. In certain embodiment, the connector plate 32 can be omitted as shown in FIGS. 2-3.

The inner surface 28 can further comprise a disinfectant 26. The disinfectant 26 may comprise a plurality of ultraviolet lights 27 wherein the ultraviolet lights can be attached to the inner surface by, for example, glue, thread, rivets, or the like wherein the inner surface can have an inner sock (not shown) attached to the inner surface for the ultraviolet lights to attach to.

The inner sock (not shown) can be manufactured from, for example, rigid or flexible plastics or metals. The ultraviolet lights 26 can be, for example, ultraviolet-A, ultraviolet-B, ultraviolet-C, or the like. In certain embodiments, the ultraviolet lights 26 can be omitted. In embodiments, the front portion 14 can be substantially conform to a mask shape 24 or it can be, for example, a clamshell shape, an egg shape, dome shape, or the like wherein the shape of the front portion can create a chamber for the mask to sit in. The front portion 14 can be manufactured from any anti-microbial plastic or metal.

The disinfectant 26 can have a controller that has a timer which can alert the user when the mask 24 is decontaminated or when the mask needs to be thrown away. The user can be alerted via visible lighting such as red, green, or orange or through a portable computing device. The user can reset the mask carrying system 10 when replaced with new mask 24. In certain embodiments the decontamination can be by chlorine capsule, plasma, coronal discharge, ultrasound, or the like. The mask carrying system 10 can be dock in a charging station (not shown) wherein the user can dock the old case and pick up a new case while that other case is be decontaminated.

In another embodiment, the disinfectant 26 can be a heater on the inner surface 28 that heats the interior of the housing to at least 167 degrees Fahrenheit for at least 30 minutes. In yet another embodiment, the disinfectant 26 can be a spray or vaporized chemical disinfectant that dispenses and/or circulates a chemical disinfectant within the interior of the housing. The disinfectant 26 may also be some combination of heat, ultraviolet light, and a chemical disinfectant. The disinfectant 26 may be calibrated to provide a particular reduction in the quantity of microbes. By way of example, the disinfectant 26 may be set to reduce the quantity of microbes on the mask 24 by a desired log reduction. For example, the disinfectant 26 may be calibrated to provide a 6-log reduction, or, in other words, be 99.9999% effective. The quantity of microbes should be reduced by at least a 2-log reduction or destroy 99.9% of a targeted pathogen. A particular scent may be added to the interior of the housing when the housing 12 is used or when the disinfection of the item inside the housing is complete to alert the user that the disinfection process is complete.

The back portion 16 can be made of rigid or flexible material and take on substantially the same radius of the user's waist or can be substantially flat. The back portion 16 can be manufactured from, for example, plastics, metals, nylon, neoprene or the like. The front portion 14 can be detachably coupled to the back portion 16 by for example, a zipper, at least one magnets, buckles, snap fasteners, hook and loop, studs, eyelets, or the like. The back portion 16 can be removably attach to the front portion 14 wherein the user can easily and safely place the mask 24 into the interior of the housing and then cover the mask either partially or completely. The interior of the housing can protect the mask 24 so that it does not become damaged. In certain embodiment, the back portion 16 can be partially attached to the front portion 14 by a flap 52, as shown in FIG. 3, which can allow the back portion to stay connected to the front portion when the user places the mask 24 within the front portion.

The back portion 16 can further comprise at least one loop 20 that can allow the housing 12 to be removably attached to a belt 22 as shown in FIG. 1, FIG. 7 and FIG. 9. The loop 20 can be, for example, a belt clip, a belt loop, a ribbon clip, a folded loop, a flat loop, integral loop, steel loop, plastic loop or the like and in certain embodiments the loop can be omitted. The belt 22 can be attached to the loop 20, as shown in FIG. 2, or it can be attached to, for example, a belt on pants, a waistline, a strap, or the like. The loop 20 can allow the housing 12 to be a clip-on attachment which can carry the mask 24 and can enable effective decontamination of the mask when applied to the user's face. The loop 20 allows for the housing 12 to be easily attached to, for example, belt, pants waistline, shoulder strap or the like and can allow the housing to be easily installed and removed from the user. The loop 20 can be attached to, for example, back, side, top, or bottom of the back portion 16. The loop 20 can be attached to the back portion 16 by, for example, rivets, glue, thread, or the like, or it can be formed to the back portion 16 as shown in FIG. 7. In certain embodiments, there can be one, two, three, four, or the like loops attached to the back portion 16 as shown in FIG. 9.

The mask carrying system 10 can further comprise a power source 36 which can be coupled or connected to the connector plate 32 wherein the wire leads (not shown) from the ultraviolet lights 26 can pass through the connector plate, or the connector plate can have a wire connector (not shown) that the ultraviolet lights are connected which allows the power source to permanently or removably connect to the ultraviolet light leads. The power source 36 can supply power to the ultraviolet lights 26 at different intensities. The power source 36 can comprise a switch which can allow the user to turn the ultraviolet lights 26 on or off, and a status light 40 allowing the user to know whether the ultraviolet lights are one or off and wither the power source needs charging or is low.

The power source 36 can be, for example, lithium-ion battery, alkaline battery, nickel cadmium battery, or the like. The power source 36 can be rechargeable and the mask carrying system 10 can be placed on a docking station (not shown) to be recharged wherein the docking station can charge the power source by contact, inductance, charging cord 42 or the like. The switch 38 can be, for example, a toggle switch, a push button switch, lever switch, slide switch or the like. The status light can be, for example, light emitting diodes, panel lights, surface mount light, or the like. The mask carrying system 10 can continue to decontamination the mask 24 while docked and can send a notification to the user via a light, or notification on a portable computing device that the mask carrying system 10 is charged and the mask is decontaminated. In certain embodiments the power source 36 can be omitted as shown in FIG. 2.

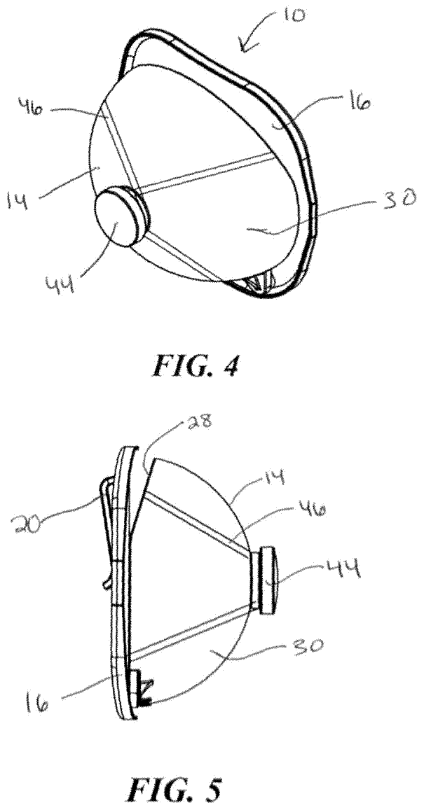

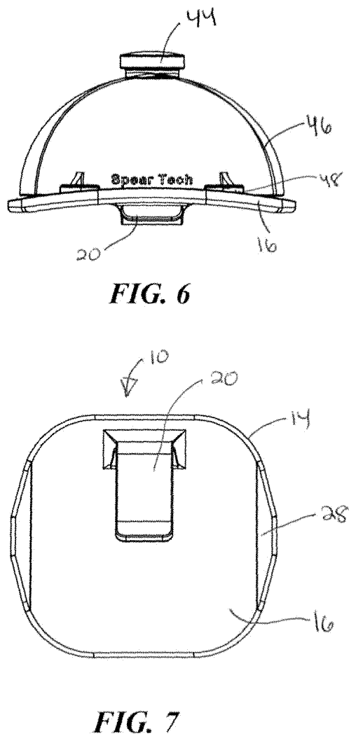

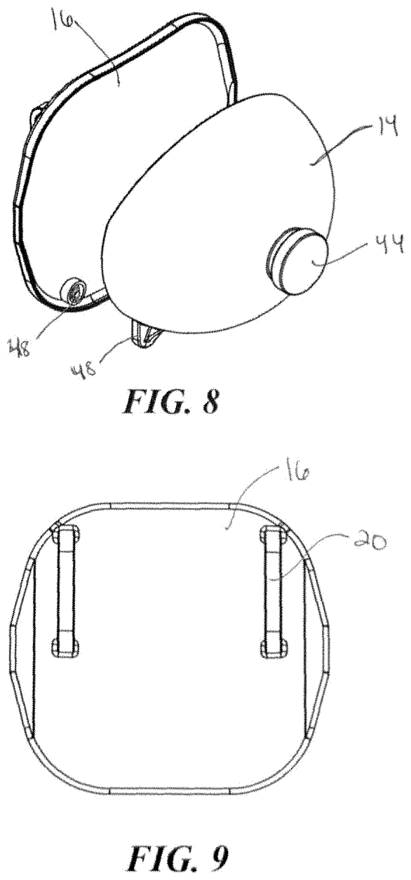

Referring to FIG. 4-FIG. 6, another embodiment of a mask carrying system 10 wherein the mask carrying system can comprise a inner surface 28 which can define the interior of the housing 12 which can grasp the contaminated side of mask 24 without directly coming into contact with the face. The mask 24 can be placed back on the face while preserving the strap position preventing the straps from contacting the dirty side of the mask. The carrier can allow the user to grasp the mask which can be a protrusion 44 from the outer surface 30 so that a user's thumb and forefinger can grasp the mask firmly by the protrusion. The mask's straps 46 can be restrained by protrusions 44 on the outer surface 30 of the housing 12 wherein the straps can slide over or can be retained by the protrusion which can be, for example, a clip, a pin, a rounded head dowel pin, captive pin, or any similar part that can hold the straps in place. The mask straps 46 can be held secure by any number of securing devices, but the preferred method is hook and loop, snaps, or the like.

In another embodiment, the inner surface 28 which defines the interior of the housing 12 can have a liner 50 which can thin, disposable plastic sheet that can be adhered to the inner surface which can be individually discarded after removal of mask or added before each use as shown in FIG. 2. The contaminated exterior of the mask 24 can be in contact with the inside of the case, viral particles can circulate throughout the interior of the housing 12 after it has been removed from the case, therefore, a single layer of the disposable plastic sheet (not shown) can protect the inner surface 28.

In another embodiment, the mask carrying system 10 can have at least one magnet 48 that can couple the front portion 14 to the back portion 16 which can allow for the back portion to be easily released from the front portion which can allow the mask 24 to be easily encapsulated. Coupling the front portion 14 with the back portion 16 can release the back portion by snapping tabs, magnets, buttons, or any similar type feature wherein it can lock the back portion onto the front portion but when pressed or pulled apart the back portion can release from the front portion and allow the user to easily remove mask from the interior of the housing 12.

A method of disinfecting an item wherein the method comprises the acts of providing a housing 12 having a front portion 14 detachably coupled to a back portion 16, and an outer surface 30 and an inner surface 28 defining an exterior and an interior of the housing. Placing the item into the interior of the housing wherein the item can be a mask 24. Coupling the front portion 14 to the back portion 16. Disinfecting the interior of the housing sufficient to provide to a 2-log reduction of at least one pathogen on the item. The act of disinfecting comprises illuminating the item with ultraviolet light using a power source which can be, for example, lithium-ion battery, alkaline battery, nickel cadmium battery, or the like.

The act of disinfecting can comprise heating the interior of the housing to at least 167 degrees Fahrenheit for at least 30 minutes. The front portion 14 can substantially conforms to a mask 24 shape and further comprises placing the mask within the housing 12 to align the shape of the mask to the contour of the front portion 14. Carrying the housing 12 coupled to a belt 22 on a user. The act of disinfecting the interior of the housing 12 comprises supplying at least one of ultraviolet light 27, heat, and a chemical disinfectant. Calibrating the amount of ultraviolet light, heat, and chemical disinfectant to provide a log-2 reduction of at least one pathogen. Docking the housing 12 onto a docking station (not shown) and recharging a power source which supplies the ultraviolet light or heat with power. Sending a notification to a user via a portable computing device when the mask is decontaminated wherein the portable computing device can be, for example, smartphone, tablet, laptop, or the like.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claims is:

1. A mask carrying system comprising:
a housing having a front portion detachably coupled to a back portion, and an outer surface and an inner surface defining an exterior and interior of the housing;
at least one loop coupled to the back portion that allows the housing to be removably attached to a belt; and
a disinfectant to disinfect an item on the interior of the housing to a 2-log reduction of at least one pathogen.

2. The system according to claim 1, further comprising a connector plate coupled to the outer surface of the front portion of the housing.

3. The system according to claim 1, wherein the disinfectant is a plurality of ultraviolet lights illuminating the interior of the housing.

4. The system according to claim 1 where in the disinfectant is a heat source that heats the interior of the housing to at least 167 degrees Fahrenheit for at least 30 minutes.

5. The system according to claim 1, wherein the front portion substantially conforms to a mask.

6. The system according to claim 2, wherein the connector plate is coupled to a power source wherein the power source supplies power to the ultraviolet lights.

7. The system according to claim 1, wherein the loop is a belt clip or a belt loop.

8. The system according to claim 1, wherein the front portion is coupled to the back portion by a zipper, at least one magnet, or a hinge and clasp.

9. The system according to claim 5, wherein the power source comprises an on/off switch and a charging port for the user to recharge the power source.

10. The system according to claim 1, wherein the belt is the user's own belt or a customized belt that is attached to the loop.

11. The system according to claim 1, wherein the front portion can further comprise a mask strap protrusion wherein the mask strap protrusion holds the straps of a mask.

12. A method of disinfecting an item, the method comprising the acts of:
providing a housing having a front portion detachably coupled to a back portion, and an outer surface and an inner surface defining an exterior and an interior of the housing;
placing the item into the interior of the housing;
coupling the front portion to the back portion;
disinfecting the interior of the housing sufficient to provide to a 2-log reduction of at least one pathogen on the item.

13. The method according to claim 12, wherein the act of disinfecting comprises illuminating the item with ultraviolet light.

14. The method according to claim 12 wherein the act of disinfecting comprises heating the interior of the housing to at least 167 degrees Fahrenheit for at least 30 minutes.

15. The method according to claim 12, wherein the front portion substantially conforms to a mask and further comprises placing the mask within the housing to align the shape of the mask to the contour of the front portion.

16. The method according to claim 12, further comprising carrying the housing coupled to a belt on a user.

17. The method according to claim 12 wherein the act of disinfecting the interior of the housing comprises supplying at least one of ultraviolet light, heat, and a chemical disinfectant.

18. The method according to claim 17, further comprising calibrating the amount of ultraviolet light, heat, and chemical disinfectant to provide a log-2 reduction of at least one pathogen.

19. The method according to claim 17, further comprising docking the housing onto a docking station and recharging a power source which supplies the ultraviolet light or heat with power.

20. The method according to claim 19, sending a notification to a user via a portable computing device when the mask is decontaminated.
